(19) United States
(12) Patent Application Publication
(10) Pub. No.: US 2025/0352540 A1
(43) Pub. Date: Nov. 20, 2025

(54) USES OF A PURE 5-HT6 RECEPTOR ANTAGONIST

(71) Applicant: SUVEN LIFE SCIENCES LIMITED, Hyderabad-Telangana (IN)

(72) Inventors: Ramakrishna NIROGI, Hyderabad (IN); Venkata Ramalingayya GRANDHI, Hyderabad (IN); Pradeep JAYARAJAN, Hyderabad (IN); Venkateswarlu JASTI, Hyderabad (IN)

(73) Assignee: SUVEN LIFE SCIENCES LIMITED, Hyderabad-Telangana (IN)

(21) Appl. No.: 19/174,476

(22) Filed: Apr. 9, 2025

Related U.S. Application Data
(63) Continuation of application No. 16/625,313, filed on Dec. 20, 2019.

(30) Foreign Application Priority Data
Jul. 3, 2017 (IN) 201741023375

(51) Int. Cl. A61K 31/496; A61P 25/28

(52) U.S. Cl. CPC A61K 31/496; A61P 25/28

(57) ABSTRACT

The present invention relates to new uses of a pure 5-HT6 receptor antagonist, specifically 1-[(2-Bromophenyl)sulfonyl]-5-methoxy-3-[(4-methyl-1-piperazinyl)methyl]-1H-indole or a pharmaceutically acceptable salts thereof, for the treatment of dementia due to menopause, senile dementia, vascular dementia, chemotherapy-induced cognitive impairment and behavioral changes in dementia such as agitation, aggression, depression, anxiety, psychosis, disinhibition, or sleep disturbances. The present invention further provides use of the said compounds in the manufacture of medicament intended for the treatment of the disorders described herein.

### FIELD OF THE INVENTION

The present invention relates to the new uses of a pure 5-HT₆ receptor antagonist, specifically 1-[(2-Bromophenyl)sulfonyl]-5-methoxy-3-[(4-methyl-1-piperazinyl)methyl]-1H-indole or a pharmaceutically acceptable salt thereof, for the treatment of dementia due to menopause, senile dementia, vascular dementia, chemotherapy-induced cognitive impairment and behavioral changes in dementia such as agitation, aggression, depression, anxiety, psychosis, disinhibition or sleep disturbances. The present invention further provides use of the said compounds in the manufacture of medicament intended for the treatment of the disorders described herein.

### BACKGROUND OF THE INVENTION

Cognitive decline occurs in women during menopause. Roughly two-thirds of women complain of forgetfulness during menopause. Empirical evidences suggests peri- and post-menopausal women performed worse on tests of memory and cognition in the year after they had their last period than in the time leading up to menopause. In peri- and post-menopausal women reached this state either naturally or by oophorectomy, cognitive function significantly declines due to chronic state of hormonal deprivation. Dementia in post-menopausal women affects various components, viz., verbal, episodic, visuo-spatial navigation along with deficits of attention and executive function which affects activities of daily living and thereby quality of life (QOL) negatively. The decline in memory is usually most pronounced within 12 months after the final menstrual period. For the majority of women, cognitive function is not likely to worsen in post-menopause in any pattern other than that expected with normal aging. Although it is not likely that in post-menopause, a woman's cognitive function will return to what it was pre-menopause, they may adapt to and compensate for the symptoms with time.

Hormone replacement therapy (HRT) was once considered to be the first line treatment strategy in post-menopause women for the abnormal physiological changes and disabilities that are unavoidable. However, the recent studies conducted largest ever in menopausal women (WHIMS-Women's Health Initiative Memory Study) concluded the negative effects of HRT along with an increased risk of uterine, ovarian and breast cancers, pulmonary embolism, cardiac disease and stroke (JAMA, 2004, 291, 47-53, JAMA, 2002, 288, 321-333). No alternative and effective therapy is approved till date in this population although some of the cholinesterase inhibitors have been tested clinically. In general, women spend one third of their life time in a state of chronic hormonal deprivation, i.e. menopause considering an age of 50 years where they undergo menopause transition.

Donepezil, a cholinesterase inhibitor which works by increasing the brain acetylcholine levels, was no more effective than placebo in treating the symptoms of menopause related memory and cognitive loss (Gender Medicine, 2007, 4, 352-358). Therefore, there is an unmet need for treating dementia due to menopause in aged women population to improve their QOL.

Serotonin-6 (5-HT₆) receptor antagonist also works by increasing the brain acetylcholine levels. Surprisingly, 5-HT₆ receptor antagonist of the present invention reversed the memory deficits in an animal model of menopause. Thus, 5-HT₆ receptor antagonist of the present invention could be a potential drug candidate for the treatment of menopause related memory and cognitive loss.

Senile dementia is a disease caused by degeneration of the brain cells and is characterized by a decrease in cognitive abilities. This may include the person's ability to concentrate, to recall information, and to properly judge a situation. Senility is a deterioration of body and mind associated with advanced aging. Indications of old age vary in the time of their appearance. Surprisingly, 5-HT₆ receptor antagonist of the present invention improved the memory in an animal model of senility.

Vascular dementia is a cognitive dysfunctional syndrome caused by various cerebral vascular diseases and it is the second most common type of dementia following Alzheimer's disease (Lancet. 2015; 386(10004):1698-706). Surprisingly, 5-HT₆ receptor antagonist of the present invention improved the memory in an animal model of vascular dementia.

Agitation, aggression, depression, anxiety, psychosis, disinhibition, sleep disturbances are the common behavioral changes in dementia patients. These behavioral changes cause great agony to dementia patients and caregivers. Therefore, there remains an unmet medical need for the treatment of the behavioral changes in dementia. The 5-HT₆ receptor antagonist of the present invention surprisingly decreases the aggression levels and hence could be the potential treatment for behavioral changes in dementia such as agitation, aggression, depression, anxiety, psychosis, disinhibition or sleep disturbances.

Cancer is a group of diseases characterized by uncontrolled growth and dissemination of abnormal cells, which is second leading cause of death globally following cardiovascular diseases. With the improved cancer survival rates globally with the advanced therapeutic strategies, research focus has been turned towards “cancer survivorship” for improving the QOL in global cancer survivors. For many patients afflicted with malignancies or cancer, chemotherapy offers the best option for disease control. Even before opting surgical and radiation procedures in cancer therapy, chemotherapy is an invaluable tool to lessen the burden of cancer and moreover, it is suggested for a more fruitful out come with the above procedures. Though chemotherapy is an effective way to treat many types of cancer, it also carries negative side effects. Due to non-specific nature of cell killing by chemotherapy, neuronal cell are not an exception that underlies the neurobiology of “chemobrain” and the associated cognitive deficits. Patients treated with chemotherapy are at an increased risk of altered brain structure and function. Clinical studies indicated that up to 70% of cancer patients who received chemotherapy experience cognitive impairment (Clin Cancer Res 18(7):1954-1965). This cognitive impairment, commonly named “chemobrain,” can affect working memory, attention, processing speed, concentration and executive functions. Deficits observed with “chemobrain” are long lasting, even up to 10 years from the last chemo received. Till date, no therapeutic intervention is available or approved for global cancer survivor population. Surprisingly, 5-HT₆ receptor antagonist of the present invention improved the memory in an animal model of memory deficits associated with chemotherapy.

Currently no drug is approved for the treatment of dementia due to menopause, senile dementia, vascular dementia, chemotherapy-induced cognitive impairment and behavioral changes such as agitation or aggression in dementia. These cognitive and behavioral changes cause great agony to patients suffering from cognitive impairment and caregivers. Therefore, there remains an unmet medical need for the treatment of dementia due to menopause, senile dementia, vascular dementia, chemotherapy-induced cognitive impairment and behavioral changes such as agitation or aggression in dementia. Surprisingly, the 5-HT₆ receptor antagonist of the present invention significantly reversed the memory deficits in various animal models indicating that it could be a potential drug candidate for the treatment of menopause related memory and cognitive loss, senile dementia, vascular dementia, chemotherapy-induced cognitive impairment and behavioral changes in dementia.

### SUMMARY OF THE INVENTION

The objective of the present invention is to provide methods for treating dementia due to menopause, senile dementia, vascular dementia, chemotherapy-induced cognitive impairment and behavioral changes in dementia.

In first aspect, the present invention relates to a method of treating dementia due to menopause, senile dementia, vascular dementia, chemotherapy-induced cognitive impairment and behavioral changes in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another aspect, the present invention relates to a method of treating dementia due to menopause comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another aspect, the present invention relates to a method of treating senile dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another aspect, the present invention relates to a method of treating vascular dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another aspect, the present invention relates to a method of treating chemotherapy-induced cognitive impairment comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another aspect, the present invention relates to a method of treating behavioral changes in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another aspect, the present invention relates to use of the pure 5-HT₆ receptor antagonist, specifically 1-[(2-Bromophenyl)sulfonyl]-5-methoxy-3-[(4-methyl-1-piperazinyl)methyl]-1H-indole or a pharmaceutically acceptable salt thereof, for the treatment of dementia due to menopause, senile dementia, vascular dementia, chemotherapy-induced cognitive impairment and behavioral changes in dementia such as agitation, aggression, depression, anxiety, psychosis, disinhibition or sleep disturbances.

In another aspect, the present invention relates to a pharmaceutical composition for use in treating dementia due to menopause, senile dementia, vascular dementia, chemotherapy-induced cognitive impairment and behavioral changes in dementia comprising a pure 5-HT₆ receptor antagonist, 1-[(2-Bromophenyl)sulfonyl]-5-methoxy-3-[(4-methyl-1-piperazinyl)methyl]-1H-indole or a pharmaceutically acceptable salt thereof and pharmaceutically acceptable excipients thereof.

### BRIEF DESCRIPTION OF ACCOMPANYING DRAWINGS

FIG. 1 depicts the effect of SUVN-502 on cognition enhancing properties using object recognition task in ovariectomized rats

FIG. 2 depicts the effect of donepezil on cognition enhancing properties using object recognition task in ovariectomized rats

FIG. 3 depicts the effect of SUVN-502 on memory deficits associated with normal ageing in Morris water maze task

FIG. 4 depicts the effect of SUVN-502 on aggressive levels in CD1 mice

FIG. 5 depicts the effect of SUVN-502 on memory deficits associated with bilateral common carotid artery ligated rats

FIG. 6 depicts the effect of SUVN-502 on memory deficits associated with DOX-induced chemotherapy-induced cognitive impairment.

### DETAILED DESCRIPTION OF THE INVENTION

Unless otherwise stated, the following terms used in the specification and claims have the meanings given below:

The term, “5-HT₆ receptor antagonist” as used herein refers to a ligand or drug that has affinity towards 5-HT₆ receptor, blocks or inhibits the function/binding of agonist at the 5-HT₆ receptor.

The term, “pure 5-HT₆ receptor antagonist” as used herein refers to 5-HT₆ receptor antagonist which has very high selectivity (>250 fold) over closely related serotonin subtypes like 5-HT_{1A}, 5-HT_{1B}, 5-HT_{1D}, 5-HT_{2A}, 5-HT_{2C}, 5-HT₄, 5-HT_{5A} and 5-HT₇.

Example of the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

Examples of pharmaceutically acceptable salt of the above identified compound include but not limited to, 1-[(2-Bromophenyl)sulfonyl]-5-methoxy-3-[(4-methyl-1-piperazinyl)methyl]-1H-indole dimesylate monohydrate.

The term, “dementia due to menopause” as used herein refers to cognitive decline, memory loss, forgetfulness or memory impairment in a peri-menopausal or post-menopausal or ovariectomized female population.

The term, “senile dementia” as used herein refers to dementia due to natural aging that occurs in aged population.

The term, “vascular dementia” as used herein refers to acute loss of memory, resulting from ischemic, ischemic-hypoxic or hemorrhagic brain lesions as a result of cardiovascular diseases and cardiovascular pathologic changes.

The term, “chemotherapy-induced cognitive impairment” as used herein refers to chemobrain, means cognitive changes that occur as a side effect of chemotherapy. These changes may be temporary changes in memory and the thinking process. Chemotherapy-induced cognitive impairment typically involves one or more of the following symptoms, difficulty in concentrating and thinking and multi-tasking, decreased memory, shortened attention span and/or feelings of disorganization. Chemotherapy-induced cognitive impairment may result from a wide variety of chemotherapeutics.

The term “behavioral changes in dementia” refer to agitation, aggression, depression, anxiety, psychosis, disinhibition, and/or sleep disturbances due to dementia. It also refers to any physical or verbal behavior of dementia patients which has the effect of hurting or repelling others, and includes aggressive behaviors such as beating, kicking, biting and screaming. The behavioral changes in dementia include the behavioral changes in Alzheimer's disease, Parkinson's disease, lewy body dementia (LBD), vascular dementia and frontotemporal dementia (FTD). Preferably, the behavioral changes in dementia is selected from aggression in Alzheimer's disease, agitation in Alzheimer's disease, anxiety in Alzheimer's disease, sleep disturbances in Alzheimer's disease, aggression in Parkinson's disease, agitation in Parkinson's disease, anxiety in Parkinson's disease and sleep disturbances in Parkinson's disease.

The phrase, “therapeutically effective amount” is defined as an amount of a compound of the present invention that (i) treats the particular disease, condition or disorder, (ii) eliminates one or more symptoms of the particular disease, condition or disorder and (iii) delays the onset of one or more symptoms of the particular disease, condition or disorder described herein.

The term, “pharmaceutically acceptable salt” as used herein refers to salts of the active compound and are prepared by reaction with the appropriate organic or inorganic acid or acid derivative, depending on the particular substituents found on the compounds described herein. The pharmaceutically acceptable salt includes but not limited to, dimesylate, dihydrochloride salt, oxalate salt, succinate, tartrate salt and the like. Preferably, the pharmaceutically acceptable salt is dihydrochloride and dimesylate salts. More preferably, the pharmaceutically acceptable salt is dimesylate salt.

The term, “patient” as used herein refers to an animal. Preferably the term “patient” refers to mammal. The term mammal includes animals such as mice, rats, dogs, rabbits, pigs, monkeys, horses and human. More preferably the patient is human.

The compound, SUVN-502 as used herein is 1-[(2-Bromophenyl)sulfonyl]-5-methoxy-3-[(4-methyl-1-piperazinyl)methyl]-1H-indole dimesylate monohydrate which has the chemical structure;

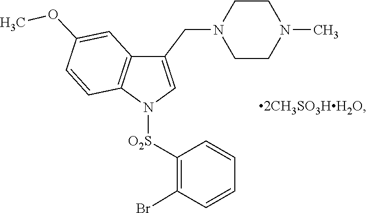

The compound, SUVN-502 and its preparation has been described in U.S. Pat. Nos. 7,875,605 and 9,540,321 respectively.

The term, “treatment’ or ‘treating” as used herein refers to any treatment of a disease in a mammal, including: (a) slowing or arresting the development of clinical symptoms; and/or (b) causing the regression of clinical symptoms.

The term, “compound for use” as used herein embrace any one or more of the following: (1) use of a compound, (2) method of use of a compound, (3) use in the treatment of, (4) the use for the manufacture of pharmaceutical composition/medicament for treatment/treating or (5) method of treatment/treating/preventing/reducing/inhibiting comprising administering an effective amount of the active compound to a patient in need thereof.

### EMBODIMENTS

The present invention encompasses all the examples described herein without limitation, however, preferred aspects and elements of the invention are discussed herein in the form of the following embodiments.

In one embodiment, the present invention relates to the method of treating dementia due to menopause, senile dementia, vascular dementia, chemotherapy-induced cognitive impairment, and behavioral changes in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating dementia due to menopause comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating senile dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating vascular dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating chemotherapy-induced cognitive impairment comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating behavioral changes in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating behavioral changes in dementia selected from agitation, aggression, depression, anxiety, psychosis, disinhibition and/or sleep disturbances comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treatment of aggression in dementia, agitation in dementia, anxiety in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating aggression in Alzheimer's disease, agitation in Alzheimer's disease, anxiety in Alzheimer's disease, aggression in Parkinson's disease, agitation in Parkinson's disease and anxiety in Parkinson's disease comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating dementia due to menopause, senile dementia, vascular dementia, chemotherapy-induced cognitive impairment and behavioral changes in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indoledimesylate monohydrate.

In another embodiment, the present invention relates to the method of treating dementia due to menopause comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole dimesylate monohydrate.

In another embodiment, the present invention relates to the method of treating senile dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole dimesylate monohydrate.

In another embodiment, the present invention relates to the method of treating vascular dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole dimesylate monohydrate.

In another embodiment, the present invention relates to the method of treating chemotherapy-induced cognitive impairment comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-11H-indole dimesylate monohydrate.

In another embodiment, the present invention relates to the method of treating behavioral changes in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole dimesylate monohydrate.

In another embodiment, the present invention relates to the method of treating behavioral changes in dementia selected agitation, aggression, depression, anxiety, psychosis, disinhibition and/or sleep disturbances comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole dimesylate monohydrate.

In another embodiment, the present invention relates to the method of treatment of aggression in dementia, agitation in dementia, anxiety in dementia comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indole or a pharmaceutically acceptable salt thereof.

In another embodiment, the present invention relates to the method of treating aggression in Alzheimer's disease, agitation in Alzheimer's disease, anxiety in Alzheimer's disease, aggression in Parkinson's disease, agitation in Parkinson's disease and anxiety in Parkinson's disease comprising administering to a patient in need thereof, a therapeutically effective amount of a pure 5-HT₆ receptor antagonist, wherein the pure 5-HT₆ receptor antagonist is 1-[(2-Bromophenyl)sulfonyl]-5-methoxy-3-[(4-methyl-1-piperazinyl)methyl]-1H-indoledimesylate monohydrate.

In yet another embodiment, the present invention relates to use of a pure 5-HT₆ antagonist in the treatment of dementia due to menopause, senile dementia, vascular dementia, chemotherapy-induced cognitive impairment and behavioral changes in dementia, wherein the pure 5-HT₆ antagonist is 1-[(2-Bromophenyl)sulfonyl]-5-methoxy-3-[(4-methyl-1-piperazinyl)methyl]-1H-indole or a pharmaceutically acceptable salt thereof.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of dementia due to menopause, senile dementia, vascular dementia, chemotherapy-induced cognitive impairment and behavioral changes in dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of dementia due to menopause.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of senile dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of vascular dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of chemotherapy-induced cognitive impairment.

In yet another embodiment, the present invention relates to 1-[(2-Bromophenyl)sulfonyl]-5-methoxy-3-[(4-methyl-1-piperazinyl)methyl]-1H-indole or a pharmaceutically acceptable salt thereof, in the treatment of behavioral changes in dementia.

In another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of behavioral changes in dementia selected from agitation, aggression, depression, anxiety, psychosis, disinhibition and/or sleep disturbances.

In another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of aggression in dementia, agitation in dementia or anxiety in dementia.

In another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the treatment of aggression in Alzheimer's disease, agitation in Alzheimer's disease, anxiety in Alzheimer's disease, aggression in Parkinson's disease, agitation in Parkinson's disease or anxiety in Parkinson's disease.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the treatment of dementia due to menopause, senile dementia, vascular dementia, chemotherapy-induced cognitive impairment and behavioral changes in dementia.

In yet another embodiment, the present invention relates to use of 1-[(2-Bromophenyl)sulfonyl]-5-methoxy-3-[(4-methyl-1-piperazinyl)methyl]-1H-indoledimesylate monohydrate in the treatment of dementia due to menopause.

In yet another embodiment, the present invention relates to use of 1-[(2-Bromophenyl)sulfonyl]-5-methoxy-3-[(4-methyl-1-piperazinyl)methyl]-1H-indoledimesylate monohydrate in the treatment of senile dementia.

In yet another embodiment, the present invention relates to use of 1-[(2-Bromophenyl)sulfonyl]-5-methoxy-3-[(4-methyl-1-piperazinyl)methyl]-1H-indole monohydrate for treating vascular dementia.

In yet another embodiment, the present invention relates to use of 1-[(2-Bromophenyl)sulfonyl]-5-methoxy-3-[(4-methyl-1-piperazinyl)methyl]-1H-indolemonohydrate in the treatment of chemotherapy-induced cognitive impairment.

In yet another embodiment, the present invention relates to use of 1-[(2-Bromophenyl)sulfonyl]-5-methoxy-3-[(4-methyl-1-piperazinyl)methyl]-1H-indoledimesylate monohydrate for treating behavioral changes in dementia.

In another embodiment, the present invention relates to use of 1-[(2-Bromophenyl)sulfonyl]-5-methoxy-3-[(4-methyl-1-piperazinyl)methyl]-1H-indoledimesylate monohydrate in the treatment of behavioral changes in dementia selected from agitation, aggression, depression, anxiety, psychosis, disinhibition and/or sleep disturbances.

In another embodiment, the present invention relates to use of 1-[(2-Bromophenyl)sulfonyl]-5-methoxy-3-[(4-methyl-1-piperazinyl)methyl]-1H-indoledimesylate monohydrate in the treatment of aggression in dementia, agitation in dementia or anxiety in dementia.

In another embodiment, the present invention relates to use of 1-[(2-Bromophenyl)sulfonyl]-5-methoxy-3-[(4-methyl-1-piperazinyl)methyl]-1H-indole dimesylate monohydrate in the treatment of aggression in Alzheimer's disease, agitation in Alzheimer's disease, anxiety in Alzheimer's disease, aggression in Parkinson's disease, agitation in Parkinson's disease or anxiety in Parkinson's disease.

In yet another embodiment, the present invention relates to use of a pure 5-HT₆ antagonist in the manufacture of a medicament in the treatment of dementia due to menopause, senile dementia, vascular dementia, chemotherapy-induced cognitive impairment and behavioral changes in dementia, wherein the pure 5-HT₆ antagonist is a compound, 1-[(2-Bromophenyl)sulfonyl]-5-methoxy-3-[(4-methyl-1-piperazinyl)methyl]-1H-indole or a pharmaceutically acceptable salt thereof.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for the treatment of dementia due to menopause, senile dementia, vascular dementia, chemotherapy-induced cognitive impairment and behavioral changes in dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for the treatment of dementia due to menopause.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for the treatment of senile dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for treatment of vascular dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for treatment of chemotherapy-induced cognitive impairment.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for the treatment of behavioral changes in dementia.

In yet another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for treatment of behavioral changes in dementia selected from agitation, aggression, depression, anxiety, psychosis, disinhibition and/or sleep disturbances.

In another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof, in the manufacture of a medicament for treatment of aggression in dementia, agitation in dementia or anxiety in dementia.

In another embodiment, the present invention relates to use of 1-[(2-Bromophenyl)sulfonyl]-5-methoxy-3-[(4-methyl-1-piperazinyl)methyl]-1H-indole or a pharmaceutically acceptable salt thereof for treatment of aggression in Alzheimer's disease, agitation in Alzheimer's disease, anxiety in Alzheimer's disease, aggression in Parkinson's disease, agitation in Parkinson's disease or anxiety in Parkinson's disease.

In yet another embodiment, the present invention relates to use of a pure 5-HT₆ antagonist in the manufacture of a medicament for the treatment of dementia due to menopause, senile dementia, vascular dementia, chemotherapy-induced cognitive impairment and behavioral changes in dementia, wherein the pure 5-HT₆ antagonist is a compound, 1-[(2-Bromophenyl)sulfonyl]-5-methoxy-3-[(4-methyl-1-piperazinyl)methyl]-1H-indoledimesylate monohydrate.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of dementia due to menopause.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of senile dementia.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of vascular dementia.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of chemotherapy-induced cognitive impairment.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of behavioral changes in dementia.

In yet another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of behavioral changes in dementia selected from agitation, aggression, depression, anxiety, psychosis, disinhibition and/or sleep disturbances.

In yet another embodiment, the present invention relates to use of 1-[(2-Bromophenyl)sulfonyl]-5-methoxy-3-[(4-methyl-1-piperazinyl)methyl]-1H-indoledimesylate monohydrate in the manufacture of a medicament for the treatment of aggression in dementia, agitation in dementia or anxiety in dementia.

In another embodiment, the present invention relates to use of 1-[(2-Bromophenyl)sulfonyl]-5-methoxy-3-[(4-methyl-1-piperazinyl)methyl]-1H-indole dimesylate monohydrate in the manufacture of a medicament for the treatment of aggression in Alzheimer's disease, agitation in Alzheimer's disease, anxiety in Alzheimer's disease, aggression in Parkinson's disease, agitation in Parkinson's disease or anxiety in Parkinson's disease.

In another embodiment, the present invention relates to a pharmaceutical composition for use in treating dementia due to menopause, senile dementia, vascular dementia, chemotherapy-induced cognitive impairment and behavioral changes in dementia comprising a pure 5-HT₆ receptor antagonist, 1-[(2-Bromophenyl)sulfonyl]-5-methoxy-3-[(4-methyl-1-piperazinyl)methyl]-1H-indole or a pharmaceutically acceptable salt thereof and pharmaceutically acceptable excipients thereof.

The pharmaceutical compositions of the present invention may be formulated in a conventional manner using one or more pharmaceutically acceptable excipients. The pharmaceutically acceptable excipients are diluents, disintegrants, binders, lubricants, glidants, polymers, coating agents, solvents, cosolvents, preservatives, wetting agents, thickening agents, antifoaming agents, sweetening agents, flavouring agents, antioxidants, colorants, solubilizers, plasticizer, dispersing agents and the like. Excipients are selected from microcrystalline cellulose, mannitol, lactose, pregelatinized starch, sodium starch glycolate, corn starch or derivatives thereof, povidone, crospovidone, calcium stearate, glyceryl monostearate, glyceryl palmitostearate, talc, colloidal silicone dioxide, magnesium stearate, sodium lauryl sulfate, sodium stearyl fumarate, zinc stearate, stearic acid or hydrogenated vegetable oil, gum arabica, magnesia, glucose, fats, waxes, natural or hardened oils, water, physiological sodium chloride solution or alcohols, for example, ethanol, propanol or glycerol, sugar solutions, such as glucose solutions or mannitol solutions and the like or a mixture of the various excipients.

In yet another aspect, the active compounds of the invention may be formulated in the form of pills, tablets, coated tablets, capsules, powder, granules, pellets, patches, implants, films, liquids, semi-solids, gels, aerosols, emulsions, elixirs and the like. Such pharmaceutical compositions and processes for preparing same are well known in the art.

In yet another aspect, the pharmaceutical composition of the instant invention contains 1 to 90%, 5 to 75% and 10 to 60% by weight of the compound of the instant invention or pharmaceutically acceptable salt thereof. The amount of the active compound or its pharmaceutically acceptable salt in the pharmaceutical composition(s) can range from about 1 mg to about 500 mg or from about 5 mg to about 400 mg or from about 5 mg to about 250 mg or from about 7 mg to about 150 mg or in any range falling within the broader range of 1 mg to 500 mg.

### EXAMPLES

The examples given below are provided by the way of illustration only and therefore should not be construed to limit the scope of the invention.

### Abbreviations

5-HT_{1A}: 5-Hydroxytryptamine 1A receptor
5-HT_{1B}: 5-Hydroxytryptamine 1B receptor
5-HT_{1D}: 5-Hydroxytryptamine 1D receptor
5-HT_{2A}: 5-Hydroxytryptamine 2A receptor
5-HT_{2C}: 5-Hydroxytryptamine 2C receptor
5-HT₄: 5-Hydroxytryptamine 4 receptor
5-HT_{5A}: 5-Hydroxytryptamine 5A receptor
5-HT₆: 5-Hydroxytryptamine 6 receptor
5-HT₇: 5-Hydroxytryptamine 7 receptor
ANOVA: Analysis of variance
BCCL: Bilateral common carotid artery ligation
cAMP: Cyclic adenosine monophosphate
CD1: Cluster of differentiation 1
EC₅₀: Half maximal effective concentration
EDTA: Ethylenediaminetetraacetic acid
GPCR: G-Protein Coupled Receptor
HCl: Hydrochloric acid
h: Hour (s)
i.p.: Intraperitoneal
i.v.: Intravenous
i.m: Intramuscular
K_{b}: Binding constant
Kᵢ: Inhibitory constant
mg: Milligram
MgCl₂: Magnesium chloride
min: Minute (s)
mM: Millimolar
nmol/L: Nanomoles per litre
nM: Nanomolar
p.o.: Per oral
s.c.: Subcutaneous
S.E.M.: Standard error of the mean
μM: Micromolar

### Example 1

Determination of K_{b} values at 5-HT₆ Receptor:

A stable CHO cell line expressing recombinant human 5-HT₆ receptor and pCRE-Luc reporter system was used for cell-based assay. The assay offers a non-radioactive based approach to determine binding of a compound to GPCRs. In this specific assay, the level of intracellular cAMP which is modulated by activation or inhibition of the receptor is measured. The recombinant cells harbor luciferase reporter gene under the control of cAMP response element.

The above cells were grown in 96 well clear bottom white plates in Hams F12 medium containing 10% fetal bovine serum (FBS). Prior to the addition of compounds or standard agonist, cells were serum starved overnight. Increasing concentrations of test compound were added along with 10 μM of serotonin in OptiMEM medium to the cells. The incubation was continued at 37° C. in CO₂ incubator for 4 hours. Medium was removed and cells were washed with phosphate buffered saline. The cells were lysed and luciferase activity was measured in a Luminometer. Luminescence units were plotted against the compound concentrations using Graphpad software. EC₅₀ values of the compound were defined as the concentration required in reducing the luciferase activity by 50%. The K_{b} values were calculated by feeding the concentration of agonist used in the assay and its EC₅₀ value in the same software.
Reference: Molecular Brain Research, 2001, 90, 110-117 and British Journal of Pharmacology, 2006, 148, 1133-1143.

### Results

SUVN-502 exhibits antagonistic activity in CRE-Luc based reporter gene assay on human recombinant 5-HT₆ receptor with no detectable agonist activity. The K_{b} value of SUVN-502 is 4.2±0.9 nM.

### Example 2

Determination of Kᵢ value at 5-HT₆ Receptor

Compound was tested at MDS pharma services and Novascreen according to the following procedures.

### Materials and Methods

Receptor source: Human recombinant expressed in Hela cells
Radioligand: [³H]-LSD (60-80 Ci/mmol)
Final ligand concentration—[1.5 nM]
Non-Specific Ligand: 5 μM Serotonin (5-HT)
Reference compound: Methiothepin mesylate
Positive control: Methiothepin mesylate
Incubation conditions: Reactions were carried out in 50 mMTris-HCl (pH 7.4) containing 10 mM MgCl₂, 0.5 mM EDTA for 60 minutes at 37° C. The reaction was terminated by rapid vacuum filtration onto the glass fiber filters. Radioactivity trapped onto the filters was determined and compared to the control values in order to ascertain any interactions of the test compound(s) with the cloned serotonin 5-HT₆ binding site.
Reference: Molecular Pharmacology, 1993, 43, 320-327.

### Results

SUVN-502 selectively binds to 5-HT₆ receptor when tested by the in-vitro radioligand binding technique on human recombinant 5-HT₆ receptor. The Kᵢ value of SUVN-502 is 2.04 nM.

### Example 3

Determination of Kᵢ value at 5-HT_{2A} Receptor:

Compound was tested according to the following procedures.

### Materials and Methods

Receptor source: Recombinant mammalian cells
Radioligand: [³H]-Ketanserine (47.3 Ci/mmol)
Final ligand concentration—[1.75 nM]
Non-Specific Ligand: 0.1 mM 1-Naphthylpiperazine (1-NP)
Reference compound: 1-Naphthylpiperazine (1-NP)
Positive control: 1-Naphthylpiperazine (1-NP)
Incubation conditions: Reactions were carried out in 67 mM Tris-HCl (pH 7.4) for 1 hour at 37° C. The reaction was terminated by rapid vacuum filtration onto the glass fiber filters. Radioactivity trapped onto the filters was determined and compared to the control values in order to ascertain any interactions of the test compound with the cloned serotonin 5-HT_{2A} binding site.
Reference: Methods in Molecular Biology, 2002, 190, 31-49

### Results

SUVN-502 binds weakly to 5-HT_{2A} receptor when tested by the in vitro radioligand binding technique on human recombinant 5-HT_{2A} receptor. The Kᵢ value of SUVN-502 is 2514±377 nM.

### Example 4

### Object Recognition Task (In Vivo Model for Dementia Due to Menopause)

Bilateral ovariectomy surgery was carried out in 7-8 weeks old female rats. Briefly, animals were anesthetized using Avertin (2,2,2-tri bromo ethanol) at 150 mg/kg, i.p. and were lay down on the surgery table. A midline incision was given on the dorsal region below the rib cage and 1 cm lateral to the either side of midline, a small incision was given on fascia to locate the adipose fat supporting the ovaries. By slowly pulling out the fat tissue, the ovary is identified and excised following the uterine horn ligation with silk sutures. Fascia was also covered with sutures and the similar procedure was repeated on the other side. Superficial skin layers were sutured and gentamicin (15 mg/kg, s.c.) was given as antibiotic and meloxicam (1 mg/kg, i.m.) as analgesic with povidone iodine applied on superficial skin layers at last. Episodic memory which is a memory of autobiographical events contextual in relation to time, place etc. was assessed 4 weeks after the surgery using object recognition task.

Object recognition task was carried out using black circular arenas (50 cm height×50 cm diameter) made of PVC laminates. A web camera (Logitech, Webcam C930e) was mounted above the behaviour observational arenas to monitor the animals during testing period. On day 1 of object recognition task, rats were habituated to the respective circular black arenas for about 45 min and returned to the home cages. On day 2, animals were presented with two similar kinds of objects (a₁ and a₂). On day 3, rats were subjected to choice trial in which animals were presented with a copy of familiar object (a₃) and a novel object (b₁). Time spent by rats exploring either familiar or novel objects was noted and compared between the objects and within the group. Hand held stop watches and countdown timer were used to record the cumulative exploration time during experimental trials. Rats were subjected to SUVN-502 treatment (1, 3 and 10 mg/kg, p.o) one hour prior to evaluation on day 2 and 3. Discriminative index was calculated as ratio of time spent exploring the novel object divided by sum of time spent exploring the novel object and familiar object in choice trial.

### Results

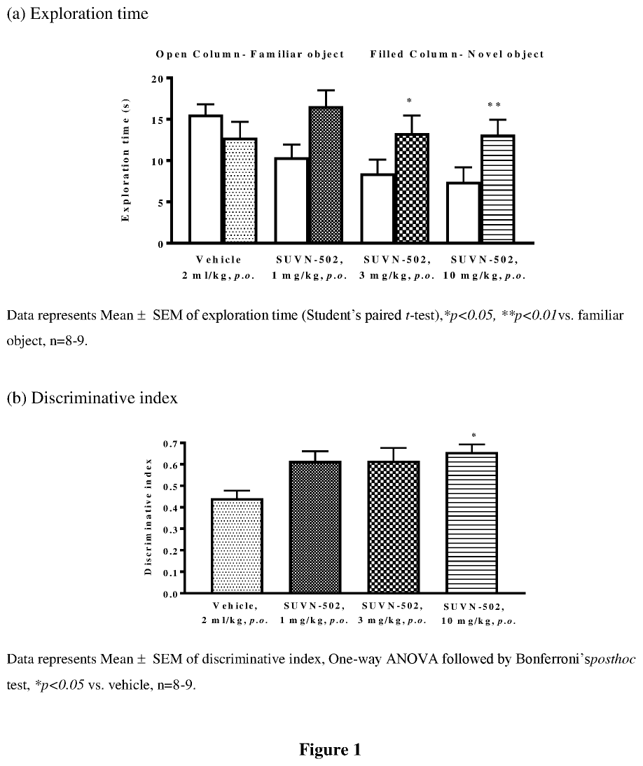

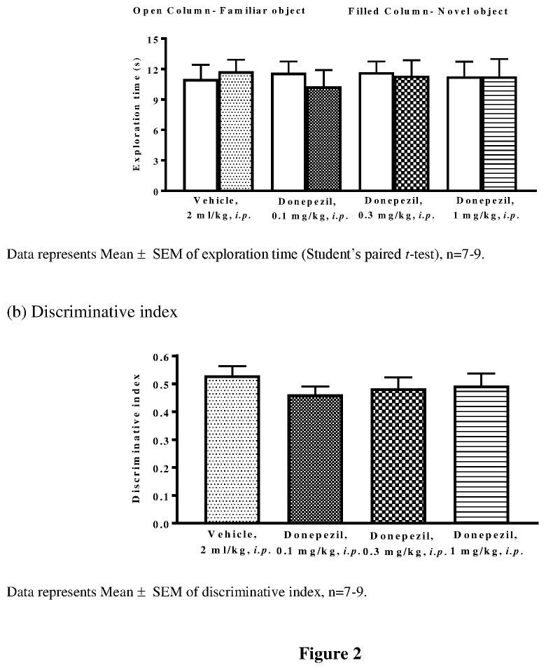

SUVN-502 reversed the object memory deficits in ovariectomized female Wistar rats in a dose dependent manner (FIGS. 1 (a) and (b)). However, donepezil did not reverse the object memory deficits in ovariectomized female Wistar rats (FIGS. 2 (a) and (b)).

### Example 5

### Morris Water Maze Task (In Vivo Model for Senile Dementia):

The water maze apparatus consisted of a circular pool (1.8 m diameter, 0.6 m high) constructed in black perspex (TSE, systems, Germany) filled with water (24° C.). The maze was positioned underneath a wide-angled video camera. A 16 cm diameter perspex platform, lying 1 cm below the water surface, was placed in the center of one of the four imaginary quadrants, which remained constant for all rats. The maze offered no intra-maze cues to guide the escape behavior, however, the training room offered several strong extra maze visual cues to aid escape learning. An automated tracking system (Videomot 2 (5.51), TSE systems, Germany) was employed to track animal and record the parameters. SUVN-502 was administered (10 mg/kg, p.o) 1 hour prior to trial, respectively.

Aged rats (˜80 weeks old) were placed facing the wall of the maze and lowered gently, feet first into water. Rats were allowed to swim for 60 seconds to find the platform. If the platform was found during this time, the trial was stopped and rat was allowed to stay on platform for 10 seconds before being removed from the maze. If the platform was not found during the 60 seconds trial, rat was guided to the platform and allowed to stay on platform for 10 seconds before being removed from the maze. The rats were taken off the platform ensuring that the rat see the investigator's hand from the front before removal. They were dried gently with a towel. Each rat received 4 trials in a day. The maze has 8 starting points. On the first, third and fifth day the animals started from 1st, 3rd, 5th and 7th starting point and on the second and fourth day the animals started from 2nd, 4th, 6th and 7th starting point. Rats were subjected to acquisition trials for 5 days. Retention of the task was assessed on 10th day, during which each animal received a single 30 seconds probe trial. The platform was removed from the pool during the trial. Rats were placed under a heating lamp for 10 min before being returned to their home cages. Latency to reach the platform (s), swim speed (cm/s) and path length (cm) were recorded during acquisition trials. Percentage time spent in target quadrant (quadrant in which platform was placed during acquisition training) and swim speed (cm/s) were recorded for the probe trial.

### Results

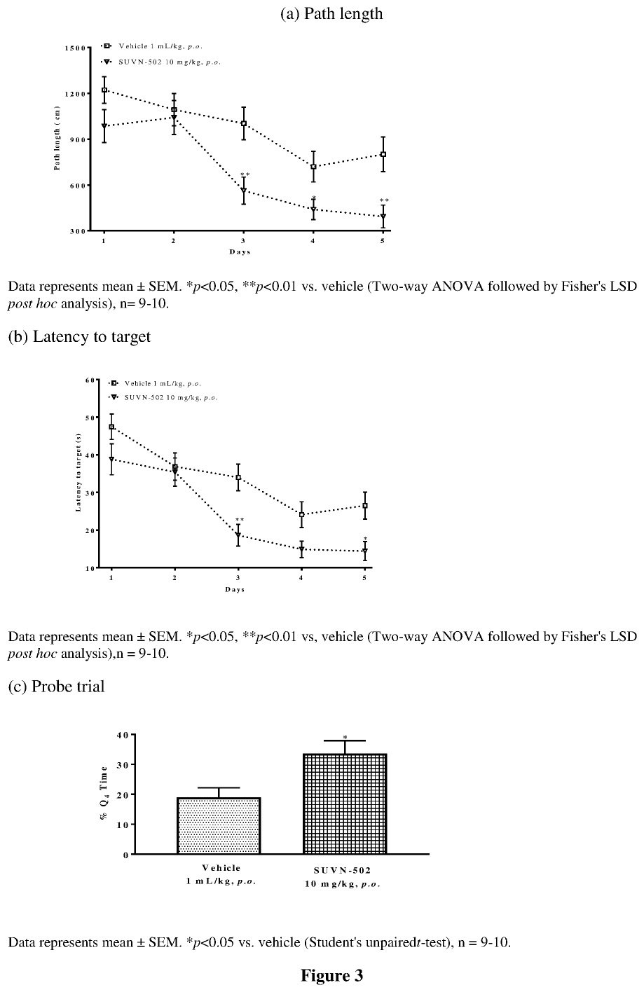

The path length of the group treated with SUVN-502 was significantly lesser (p<0.05, p<0.01) on days 3, 4 and 5 when compared to the vehicle treated group. The target latency of the group treated with SUVN-502 was also lesser on days 3, 4 and 5, however the effect reached statistical significance (p<0.05, p<0.01) only on day 3 and 5 when compared to the vehicle treated group. During the probe trial, SUVN-502 treatment group spent significantly (p<0.05) more time in the target quadrant compared to the vehicle treatment (FIGS. 3 (a), (b) and (c)).

### Example 6

### Resident Intruder Task (In Vivo Model for Aggression Due to Dementia):

Male CD1 mice of weight 20-35 g (Resident), 15-25 g (Intruder) and ovariectomized female mice (20-25 g) were used. Resident mice were habituated individually with ovariectomized female mice in each cage. β-estradiol at a dose of 0.2 mg/kg, s.c. was administered to female mice during habituation. Intruders were habituated socially for 1 week.

On day 1 and day 2, intruder was exposed to resident mice in resident's home cage for a period of 10 minutes and duration of attack was recorded. During this exposure period, female mice were removed from the cage. On day 4, animals were randomized based on their duration of attack and respective treatments were administered. SUVN-502 (1, 3 and 10 mg/kg, p.o.) and vehicle were administered to resident mice 60 minutes prior to the trial. After post dose interval, resident mice were exposed to same intruder for 10 min and duration of attack was recorded.

### Results

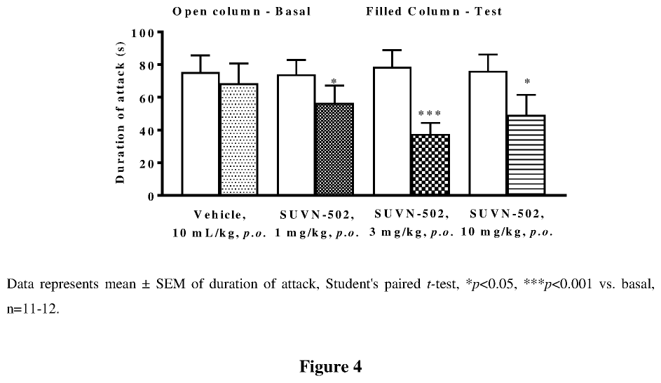

SUVN-502 decreased the aggression levels of CD1 mice at doses of 1, 3 and 10 mg/kg, p.o. (FIG. 4).

### Example 7

### Contextual Fear Conditioning (In Vivo Model for Vascular Dementia):

Male Wistar rats of age 2-3 months were used. Rats were induced vascular dementia by surgical procedure, which involves bilateral common carotid artery ligation (BCCL). Briefly, rats were anaesthetized using 2-5% isoflurane gaseous anesthesia. A dorsal incision was made near the neck region and two bilateral common carotid arteries were exposed. Both the arteries were separated from their sheaths and vagal nerves, and permanently ligated using 4-0 silk sutures. Sham animals were subjected to surgery except for ligation. After an induction period of 14 days, rats were examined in fear conditioning task. On day 1, rats were placed in the behavioral chamber and allowed to acclimatize for 1 min. Post acclimatization rats received a conditioned stimulus (CS) (tone for 10 sec) followed by an unavoidable foot shock (unconditioned stimulus (US): electric shock of 0.4 mA for 1 sec). Following a 40 sec interval between each administration, tone and shock were repeated to deliver a total of six CS-US pairings. On day 2, rats are administered with vehicle or SUVN-502. After post dose interval of 60 min, animals were scored for the duration of freezing without shock stimulus. Shocking and scoring was controlled by Freeze frame software.

### Results:

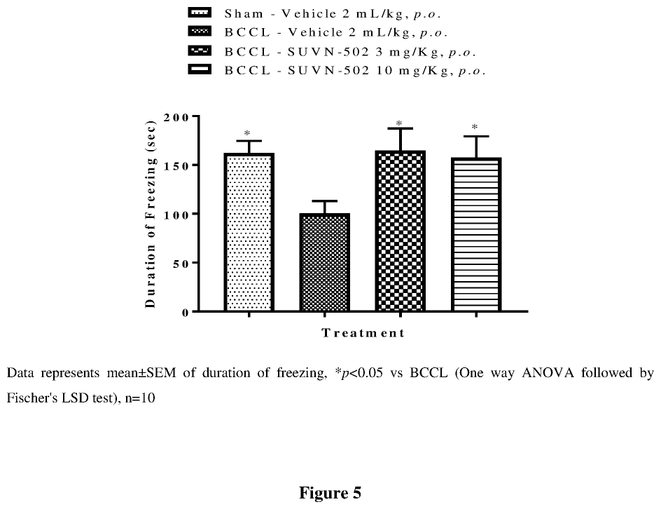

SUVN-502 improved the memory of BCCL rats at doses of 3 and 10 mg/kg, p.o. (FIG. 5).

### Example 8

### Object Recognition Task (In Vivo Model for Chemotherapy-Induced Cognitive Impairment):

The cognition-enhancing properties of SUVN-502 in deficits associated with chemotherapy were estimated using an animal model of cognition i.e., object recognition task.

Male Wistar rats (230-280 g) were used as experimental animals. Four animals were housed in each cage. Rats were acclimatized for 7 days (Days 1-7) to the laboratory conditions. Chemotherapy-induced cognitive impairment was induced by injecting doxorubicin (DOX) at 2.5 mg/kg, i.p. once in every 5 days up to 8 cycles (days 8-49). Following 4 cycles, rats were also treated with SUVN-502 at 1 and 10 mg/kg, p.o. along with DOX, i.p. The object recognition task was carried out in a 50×50 cm circular open field made up of acrylic. On experimental day 50, following 60 min of formulation dosing, animals were habituated to the arenas for 45 min. On day 51, animals were treated with their respective formulations 60 min prior to the familiarization trial (T₁) during which rats was presented with two similar objects i.e., silver Milton flasks (a₁ and a₂) for 3 min. After an interval of 30 min, rats were subjected to choice trial (T₂), with one familiar (silver, a₃) and one novel (red, b) object for a period of 3 min. During the T₁ and T₂ trials, exploration time of each object (defined as sniffing, licking, chewing or having moving vibrissae whilst directing the nose towards the object at a distance of less than 1 cm) were recorded separately by hand held stop watch.
Reference: Behavioural Brain Research, 1988, 31, 47-59.

### Results

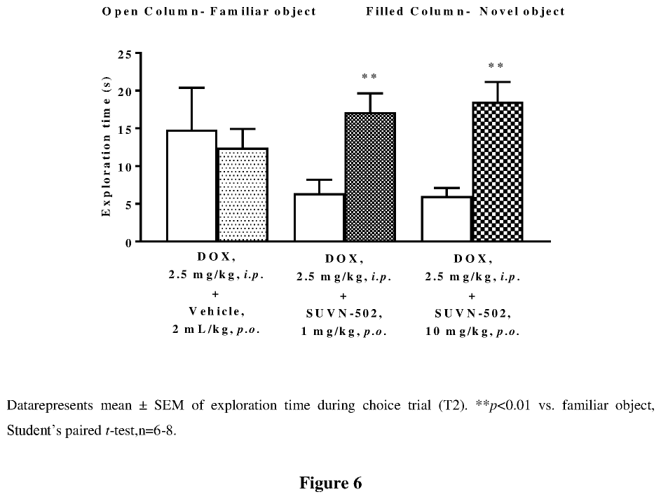

SUVN-502 has shown increased novel object recognition indicating positive effects on cognition viz; significantly higher exploration times towards the novel object relative to familiar object (FIG. 6).

1. A method of treating dementia due to menopause, senile dementia, vascular dementia, chemotherapy-induced cognitive impairment, or behavioral changes in dementia comprising administering to a patient in need thereof, a therapeutically effective amount of a pure 5-HT₆ receptor antagonist, wherein the pure 5-HT₆ receptor antagonist is a compound, 1-[(2-Bromophenyl)sulfonyl]-5-methoxy-3-[(4-methyl-1-piperazinyl)methyl]-1H-indole or a pharmaceutically acceptable salt thereof.

2. The method as claimed in claim 1, wherein the pharmaceutically acceptable salt of 1-[(2-Bromophenyl)sulfonyl]-5-methoxy-3-[(4-methyl-1-piperazinyl)methyl]-1H-indole is selected from mesylate salt, hydrochloride salt, oxalate salt, succinate or tartrate salt.

3. The method as claimed in claim 1, wherein the pharmaceutically acceptable salt of 1-[(2-Bromophenyl)sulfonyl]-5-methoxy-3-[(4-methyl-1-piperazinyl)methyl]-1H-indole is 1-[(2-Bromophenyl)sulfonyl]-5-methoxy-3-[(4-methyl-1-piperazinyl)methyl]-1H-indole dimesylate monohydrate.

4. The method of treating dementia due to menopause as claimed in claim 1, comprising administering to a patient in need thereof, a therapeutically effective amount of 1-[(2-Bromophenyl)sulfonyl]-5-methoxy-3-[(4-methyl-1-piperazinyl)methyl]-1H-indole dimesylate monohydrate.

5. The method of treating senile dementia as claimed in claim 1, comprising administering to a patient in need thereof, a therapeutically effective amount of 1-[(2-Bromophenyl)sulfonyl]-5-methoxy-3-[(4-methyl-1-piperazinyl)methyl]-1H-indole dimesylate monohydrate.

6. The method of treating vascular dementia as claimed in claim 1, comprising administering to a patient in need thereof, a therapeutically effective amount of 1-[(2-Bromophenyl)sulfonyl]-5-methoxy-3-[(4-methyl-1-piperazinyl)methyl]-1H-indole dimesylate monohydrate.

7. The method of treating chemotherapy-induced cognitive impairment as claimed in claim 1, comprising administering to a patient in need thereof, a therapeutically effective amount of 1-[(2-Bromophenyl)sulfonyl]-5-methoxy-3-[(4-methyl-1-piperazinyl)methyl]-1H-indole dimesylate monohydrate.

8. The method of treating behavioral changes in dementia as claimed in claim 1, comprising administering to a patient in need thereof, a therapeutically effective amount of 1-[(2-Bromophenyl)sulfonyl]-5-methoxy-3-[(4-methyl-1-piperazinyl)methyl]-1H-indole dimesylate monohydrate.

9. The method of treating as claimed in claim 1&, wherein the behavioral changes in dementia is selected from agitation, aggression, depression, anxiety, psychosis, disinhibition or sleep disturbances.

10. The method of treating as claimed in claim 1, wherein the behavioral change in dementia is selected from aggression in dementia, agitation in dementia or anxiety in dementia.

11. The method of treating as claimed in claim 1, wherein the behavioral changes in dementia is selected from aggression in Alzheimer's disease, agitation in Alzheimer's disease, anxiety in Alzheimer's disease, aggression in Parkinson's disease, agitation in Parkinson's disease or anxiety in Parkinson's disease.

12-23. (canceled)

24. A pharmaceutical composition comprising the pure 5-HT₆ receptor antagonist as claimed in claim 1 and pharmaceutically acceptable excipients for the treatment of dementia due to menopause, senile dementia, vascular dementia, chemotherapy-induced cognitive impairment or behavioral changes in dementia.

25. (canceled)

26. The pharmaceutical composition as claimed in claim 24, wherein the composition is to be administered to the patient by oral, nasal, local, dermal or parenteral routes.

27. The pharmaceutical composition as claimed in claim 24, wherein the composition is to be administered to the patient one to three times per day, one to three times per week or one to three times per month.
